(19) United States
(12) Patent Application Publication
(10) Pub. No.: US 2009/0306538 A1
(43) Pub. Date: Dec. 10, 2009

(54) USE OF A SCALAR VALUE TO ASSESS NEUROLOGICAL STATUS

(76) Inventor: Kamran Siminou, Newport Coast, KS (US)

Correspondence Address: MINTZ, LEVIN, COHN, FERRIS, GLOVSKY AND POPEO, P.C, ONE FINANCIAL CENTER, BOSTON, MA, 02111, US

(21) Appl. No.: 12/436,469

(22) Filed: May 6, 2009

Related U.S. Application Data
(60) Provisional application No. 61/050,974, filed on May 6, 2008.

(51) Int. Cl. A61B 3/11

(52) U.S. Cl. 600/558; 351/205

(57) ABSTRACT

Methods, systems and devices for determining whether a patient has an abnormally high level of intracranial pressure is described. The method includes using a pupillometer to obtain pupillary response data from the patient. The pupillary response data can be representative of one or more pupillary response characteristics of the patient. The method further includes providing a data analysis system comprising a microprocessor that is in communication with the pupillometer. The microprocessor includes an algorithm that converts the pupillary response data to a scalar value that is indicative of the patient's level of intracranial pressure. The microprocessor can be a stand-alone computer connected to the pupillometer or it can be integral with the pupillometer. The method further includes using the data analysis system to derive a scalar value based on the pupillary response data from the patient, wherein the scalar value is indicative of the patient's level of intracranial pressure. The scalar value can be represented by a numerical value, graphical depiction, color, sound, or other visual or audio means that indicates a value. The scalar value can be a Scalar value that indicates that the patient's pupillary response characteristics indicate that the patient's intracranial pressure is within a normal range, an abnormal range, or that the pupillary response characteristics indicate that the pupil is non-responsive.

### REFERENCE TO PRIORITY DOCUMENT

This application claims the benefit of priority under 35 U.S.C. §119(e) of U.S. Provisional Patent Application Ser. No. 61/050,974 entitled “Use of a scalar value to assess neurological status,” filed May 6, 2008. Priority of the aforementioned filing date is hereby claimed, and the full disclosure of the aforementioned application is hereby incorporated by reference in its entirety.

### FIELD OF THE INVENTION

The present invention relates generally to pupillometry systems and, more particularly, to methods and systems for assessing a patient's neurological status using a pupillometer with an output that includes a neurological pupil index (“Scalar value”).

### BACKGROUND

Incorporated herein by reference in their entirety are U.S. Pat. Nos. 7,147,327; 6,820,979; 6,260,968; 6,116,736; and 7,216,985 and all pending patent applications related thereto. The aforementioned patents relate to pupillometers that can detect irregularity in pupil size and shape, as well as capture and track pupillary dynamic responses to various types of stimuli, such as light stimulus to the eyes, as well as various forms of noxious, auditory, and other forms of stimulus. The methods and systems described herein can be used with the pupillometers described in these patents, in addition to the pupillometers described below.

Medical practitioners require multiple ways to monitor and assess the neurological status of patients who have experienced intracranial injury. This is due to the complexity of intracranial dynamics and the complex physiology of brain injury. One of the physiological characteristics that medical practitioners monitor is intra-cranial pressure (ICP). Patients with a Glasgow Coma Scale (GCS) score between 3 and 8 are candidates for ICP monitoring (Bader & Littlejohns, 2004). ICP is monitored in order to assess a patient's neurological status. Some indications for ICP monitoring include aneurismal subarachnoid hemorrhage, brain tumor, decompensated hydrocephalus, cerebral hypoxia or anoxia producing edema, traumatic brain injury, and Reyes syndrome, among others (id). Currently, ICP is monitored using an invasive procedure that requires placement of a catheter into the brain and connecting it to a pressure transducer. A significant disadvantage to the current method of monitoring ICP is that the procedure is invasive and potentially dangerous. Nonetheless, it is an important procedure, because it provides the practitioner with a means to assess the neurological status of the patient.

Neurological deterioration and intracranial injury can also be assessed by clinicians manually using a pupil gauge and a flashlight. This is a non-invasive procedure. As demonstrated below, however, there are many drawbacks to this method of assessing intracranial injury or assessing neurological status.

Different neuroanatomical pathways are involved in the control of the pupil and the integrity and functionality of these neurological pathways can often be ascertained through the analysis and interpretation of pupil behavior. This makes pupil size and the pupillary light reflex an important factor to be considered in many clinical conditions as described for example, in the work of Loewenfeld and Lowenstein, 1993. In the management and prognosis of severe traumatic brain injury, abnormalities of pupillary response or anisocoria (pupil size asymmetries) are often associated with neurologic deteriorations and they are correlated with poor neurologic outcome (Braakman et al. 1980, Choi et al. 1988, Levin et al. 1990, Marshall et al. 1991). It is also well-known that measurement of intra-cranial pressure is the gold standard for assessing neurological status (Choi et al. 1988).

More specifically, the location of the pupilomotor nuclei in the dorsal midbrain and the efferent oculomotor nerve running from the midbrain to the superior orbital fissure is particularly important for assessing an onset of descending transtentorial herniation and brainstem compression. Depressed light reflex and anisocoria have been associated with such phenomena (Goebert 1970, Manley and Larson, 2002) and they have been proposed as prognostic indicators of functional recovery after traumatic transtentorial herniation (Andrews and Pitts, 1991, Sakas et al. 1995). In addition to herniation and third nerve compression, it has been shown through blood flow imaging that pupil changes in the neurological intensive care unit are highly correlated with brain oxygenation perfusion/ischemia (Ritter et al 1999). Other investigators have used pupil size and reactivity as the fundamental parameters of more general outcome predictive models in conjunction with other clinical information such as age, mechanism of injury and Glasgow Coma Scale (GCS) (Narayan et al. 1981, Marmarou et al. 2007) and correlated such models with the presence and the location of intracranial mass lesions (Chesnut et al. 1994). It has been shown that neurosurgeons have the tendency to triage patients into conservative therapy or surgical evacuation of mass lesions depending on the pupillary status (Tien et al. 2005) and that patients promptly operated on after a new pupil abnormality have a better chance of recovery (Clusmann 2001).

Common terminology employed in the clinical literature to describe the pupil light reflex and pupil size includes “unilateral” or “bilateral unreactive pupils,” “fixed” or “dilated pupils” as well as “brisk”, “sluggish” and “nonreactive” pupils. These subjective terms are applied without a standard clinical protocol or definition. A more precise assessment of the pupil is problematic since manual pupillary assessment as part of the clinical routine is subject to compounded sources of inaccuracies and inconsistencies and is characterized by large inter-examiner variability (Litvan et al. 2000). Moreover, ambient light conditions can affect the validity of the visual assessment of the pupil and increase the inter-observer disagreement. These factors may include, for example, poor lighting conditions in the patient's room, the examiner's visual acuity, and the distance and orientation of the flashlight stimulus with respect to the patient's eye and its strength (Worthley 2000). In a recent study conducted by Du et al. 2005 (see also Meeker et al. 2005), the inter-examiner disagreement in the manual evaluation of pupillary reaction was 39%, similar to what was previously shown by Wilson et al. 1988. Thus, there is clearly a need for methods, systems, and devices for assessing with reliability the response of the pupil to light stimulus and a means of accurately correlating that response to an assessment of brain injury.

### SUMMARY

The size of the pupil and its response to light or other stimuli has been widely investigated in many different areas of medicine. In the management and prognosis of severe traumatic brain injury, deficiencies in pupillary responses are often associated with different types of intracranial neurologic deterioration and are considered important indicators for outcome. Manual examination of the pupil is affected by several compounded inaccuracies and subjectivity. Methods and systems are introduced herein that classify the pupil's response to light using a neurological pupil index (“Scalar Value™”), which can be used to determine the level of brain injury or to assess a patient's neurological status.

In one embodiment, a method for determining whether a patient has an abnormally high level of intracranial pressure is described. The method includes using a pupillometer to obtain pupillary response data from the patient. The pupillary response data can be representative of one or more pupillary response characteristics of the patient. The method further includes providing a data analysis system comprising a microprocessor that is in communication with the pupillometer. The microprocessor includes an algorithm that converts the pupillary response data to a scalar value that is indicative of the patient's level of intracranial pressure. The microprocessor can be a stand-alone computer connected to the pupillometer or it can be integral with the pupillometer. The method further includes using the data analysis system to derive a scalar value based on the pupillary response data from the patient, wherein the scalar value is indicative of the patient's level of intracranial pressure. The scalar value can be represented by a numerical value, graphical depiction, color, sound, or other visual or audio means that indicates a value. The scalar value can be a Scalar value that indicates that the patient's pupillary response characteristics indicate that the patient's intracranial pressure is within a normal range, an abnormal range, or that the pupillary response characteristics indicate that the pupil is non-responsive.

In another embodiment, a diagnostic system for assessing a patient is provided. The diagnostic system includes a pupillometer and a microprocessor that is in communication with the pupillometer. The pupillometer is capable of receiving pupillary response data representative of one or more pupillary response characteristics of said patient. The microprocessor includes an algorithm that converts said pupillary response data to a scalar value that indicates the patient's level of intracranial pressure.

In another embodiment, a pupillometer is described. The pupillometer is capable of receiving pupillary response data representative of one or more pupillary response characteristics from an eye of a patient. The pupillometer has a microprocessor with an algorithm that converts the pupillary response data to a scalar value that indicates the patient's level of intracranial pressure.

### BRIEF DESCRIPTION OF THE DRAWINGS

FIG. 1 depicts an example of a pupil light reflex profile.

FIGS. 2A-2F show the temporal progression of the Scalar value in two patients.

FIG. 3 depicts the correlation between ICP and Scalar value in patients.

FIGS. 4A-4C depicts the distribution of human error in manual pupil assessment.

FIGS. 5A-5B depict examples of pupils erroneously classified by manual assessment.

### DETAILED DESCRIPTION

Disclosed are methods and systems that provide clinicians with a scalar value that is used to assess the level of a patient's brain injury. In one embodiment, a pupillometer, such as the pupillometer described in U.S. Pat. Nos. 7,147,327; 6,820,979; 6,260,968; 6,116,736; and 7,216,985, all incorporated herein by reference in their entirety, is used to gather information about the dynamic response of a patient's pupil. The pupillometer communicates with a microprocessor, which can be a stand-alone microprocessor separate from the pupillometer, or it can be built or integrated into the pupillometer. The microprocessor can include an algorithm that transforms the pupillary response information into a scalar value. That scalar value is provided to the clinician as an output.

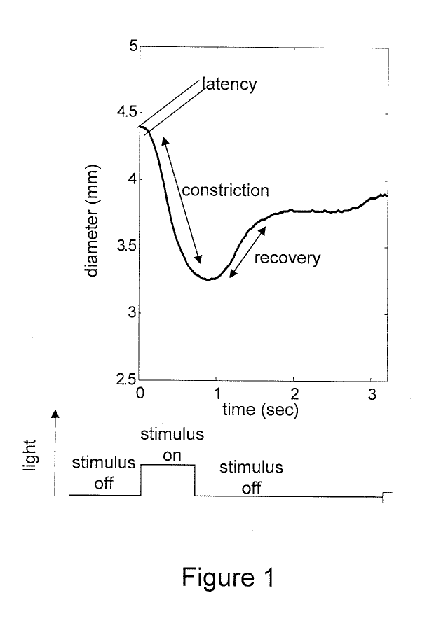

The pupillometer is capable of receiving and recording pupillary response data from a patient, said pupillary response data being representative of one or more pupillary response characteristics of a patient. The pupillary response data may be representative of various components of a pupil's response to a stimulus. For example, some of the pupillary response characteristics may include pupillary latency indicia, pupillary constriction velocity indicia, pupillary first and second dilation velocity indicia, pupillary amplitude indicia, pupillary diameter indicia, and segmental dynamic analysis indicia. For example, as shown and graphically represented in FIG. 1, some of the information that the pupillometer gathers and records is pupillary latency, velocity of the constriction and velocity of the recovery, resting pupil size before the constriction, and the amount of the constriction. This information can be depicted graphically on a display built into the pupillometer or on a display with which the pupillometer communicates. The information can also or alternatively be presented in a print-out from a printer connected to the pupillometer. The information can also or alternatively be represented by a sound emitted by a speaker built into the pupillometer or connected to the pupillometer.

Various types of stimuli may be used to stimulate the pupil. These are described in more detail in U.S. Pat. Nos. 7,147,327; 6,820,979; 6,260,968; 6,116,736; and 7,216,985, all of which are incorporated herein by reference in their entirety. In one embodiment, as described in the aforementioned patents, the stimuli is a light stimulus that is integrated into the pupillometer.

The scalar value can be represented numerically, by a sound, by a graph, by color-coding, or by any means capable of representing a value. In one embodiment, the scalar value is represented by a range of numbers and letters. A numerical range of 3-5 represents ICP in the normal range. A numerical range of 0-3 represents a larger distribution of sustained ICP. And the letters NR represents a non-reactive or no response from the pupil to the stimulus.

Clinicians' manual rating of the pupils of patients with severe traumatic brain injury and increased ICP were compared with the automated ratings given by a pupillometer with a scalar value output. The NeurOptics® ForeSite™ Pupillometer Model No. 49000 and a scalar value called NPi™ were used to rigorously assess the integrity of the pupil light neurological pathways. The results that were obtained by using the pupillometer with a scalar value output showed unexpectedly that a scalar value could be used to discriminate between those patients or subjects with elevated ICP and those with normal levels of ICP. The improvement over the accuracy in assessment of pupillary response versus manual ratings provided by experienced clinicians was also surprising.

One-hundred and seventy-two patients in the intensive care units of six different clinical sites were enrolled in a study. The neurologic status of the patients was monitored continuously for 72 hours. Pupillary examination was performed using both manual examination (flashlight for pupil reactivity and pupil gauge for pupil size) and the portable NeurOptics® pupillometer. Other information was also recorded including ICP, therapeutic interventions, concomitant medications, as well as CT scan radiologic reports.

Patients with a scalar value in the abnormal range were characterized by peaks in ICP which were significantly higher than the peaks in ICP recorded in patients with a scalar value in the normal range: averaged peak for the abnormal scalar value group was 30.5 mmHg vs. 19.9 mmHg for the normal scalar value group (p<0.00001). Patients with a scalar value indicating “nonreactive” had the highest average peak distribution of ICP (μ=35.7 mmHg). In the group of patients with an abnormal scalar value, it was found that the first event of abnormality occurred, on average, 15.9 hours prior to the time of the peak of ICP (CI=−28.56, −3 hr.) Pupil data were divided into three different groups by size; small pupils (<3 mm), medium pupils (between 3 mm and 5 mm) and large pupils (>5 mm). The mean absolute errors in pupil size measurements (manual examination with pupil gauge compared to the pupillometer reading) were respectively; μ=0.49 mm (SD 0.42) for small pupils, μ=0.71 mm (SD 0.58) for medium pupils, μ=1.07 mm (SD 0.93) for large pupils. Pupil reactivity was analyzed using the common classification of manual pupillary examination; “brisk”, “sluggish” and “nonreactive.” The manual classification, as judged by the clinicians, was evaluated against the scalar value based on the pupillometer acquisition. Manual evaluation of pupil reactivity was found to be erroneous, especially for the small pupil size range, a size which accounted for 73% of all pupils measured during the study. The rater agreement—Cohen's kappa index—between the manual evaluation and the scalar value for small, medium and large pupils was respectively, 0.1, 0.44 and 0.7.

Pupil examination is an integral part of the treatment and management protocol for severely head injured patients. This study revealed evidence that the measurement of pupil size and the evaluation of the pupillary light reflex, as subjectively evaluated during manual examination, is often erroneous, especially in small pupils which account for most of the pupils measured. A scalar value provides a rigorous and automatic metric which removes human examiner subjectivity in the evaluation of the pupil. In this study, when applied to patients with severe traumatic brain injuries, the automated pupillometer using a scalar value called NPi™ was able to discriminate those patients with elevated peaks of ICP from those with normal ICP. Automated pupillometry and the objective scale of a scalar value may benefit clinicians while managing patients with severe intracranial injury.

The portable NeurOptics infrared pupillometer was used in this study to measure the pupil size and to assess the pupil light response in patients who had sustained intracranial injury. The Neurological Pupil index (NPi™), a scalar value derived from an algorithm which uses the dynamic pupillary parameters of healthy subjects with an unaltered parasympathetic pupil light reflex pathway, provides a rigorous and non-subjective evaluation of the pupil response. A scalar value, applied to data collected with the pupillometer, was used in this study to evaluate the rate of misrepresentation of manual pupillary observations. It was also found that a scalar value was able to discriminate between patients with abnormally high ICP and patients with ICP at normal levels.

Patients were enrolled in this study from a total of six different Neurological ICU sites with a diagnosis of either traumatic brain injury (“TBI”), aneurysmal subarachnoid hemorrhage or spontaneous intracerebral hemorrhage. This group of patients typically require ICP monitoring and they usually exhibit a high potential for early changes in brain volume (ICV). A total number of 172 patients were originally enrolled. Thirty-eight of these were excluded for ocular injuries or malformation of the ocular structures found after enrollment or due to premature withdrawal from the trial (for example for family decision to withdraw support). Patients were at least 16 years of age and caregiver or next-of-kin for subjects of legal age provided informed consent for participation in the trial. Enrollment criteria required the subject to have at least one reactive pupil at the time of the enrollment and evidence of brain damage that is suggestive of raised ICP on admission CT Scan, i.e. a CT scan showing either compressed/absent cisterns, a midline shift greater than or equal to 3 mm or an intracranial mass greater than 25 cc. Patients were followed for 72 hours after the initiation of measurements. This period of time is when patients are most at risk for deterioration in their neurologic status. ICP was monitored continuously. Pupil examination with the pupillometer and ICP were recorded every 30 minutes; manual pupil examination with pupil gauge and flashlight were conducted and recorded by either the clinical research coordinator or the bedside nurse every hour. Five patients required barbiturates to manage their intracranial injuries; the barbiturate levels were monitored for the duration of the trial. For these patients, the data that were, or could possibly be affected by the drug, were excluded from analysis.

The Neuroptics® pupillometer, used in the study to evaluate pupil size and reactivity, is a hand-held infrared system which automatically tracks and analyzes pupil dynamics. Some of the aspects of the Neuroptics pupillometer are described in U.S. Pat. Nos. 7,147,327; 6,820,979; 6,260,968; 6,116,736; and 7,216,985, all of which are incorporated herein by reference in their entirety. The device is compact and easy to use which makes it well suited for challenging clinical environments such as an intensive care unit. The system is battery operated and has a color LCD screen. A measurement is initiated with the push of a button. The detection and tracking of the pupil is automatic and results are graphically displayed on the LCD screen and also saved in the device's internal memory. A detachable headrest facilitates the correct placement of the pupillometer in front of the eye and prevents microbial cross-contamination between patients. A pupil light measurement consists of a flash of light of fixed intensity and duration to stimulate the pupil light constriction reflex. The measurement lasts 3.2 s allowing a full or partial recovery of pupil size after the light constriction. The pupil is tracked at over 30 frames per second. The reliability and robustness of the pupil tracking algorithms and the corresponding pupil measurement precision and accuracy have been extensively evaluated by the manufacturer.

Pupillary characteristics are measured by the pupillometer and reported on the screen of the device at the end of each measurement. Latency is defined as the time difference between the initiation of retinal light stimulation and the onset of pupillary constriction (FIG. 1). The minimum pupil size is the pupil size at the end of the constriction. Maximum pupil size is the initial resting pupil size and is defined by the mean pupil size during the latent period. The constriction percentage is defined as maximum size minus minimum size divided by the maximum size. The constriction velocity is the amount of the constriction divided by the duration of the constriction. The dilation velocity, related to the recovery of the pupil after the constriction, is also calculated (FIG. 1).

FIG. 1 shows an example of a pupil light reflex profile. A light stimulus is flashed to the eye. The pupil is initially latent for approximately 150-300 msec and then it constricts. Once eye stimulation is terminated, pupil diameter recovers from the point of maximum constriction, slowly converging to its initial resting position. The hand-held NeurOptics® pupillometer automatically controls the stimulation and pupil data acquisition and reports, at the end of each measurement, information about latency, velocity of the constriction and recovery, resting pupil size before the constriction and the amount of the constriction.

The NPi™ is a scalar value, which is derived from an algorithm developed by NeurOptics® that uses the pupil variables defined above. A single pupil light reflex measurement is rated on a scale between 0 and 5. A scalar value score equal to or above 3 for a particular measurement means that the parameters of the reflex fall within the boundaries of normal pupil behavior. A scalar value below 3 means the reflex is abnormal, i.e., weaker than a normal pupil response as defined. The absence of even a constriction is interpreted as a nonreactive scalar value score.

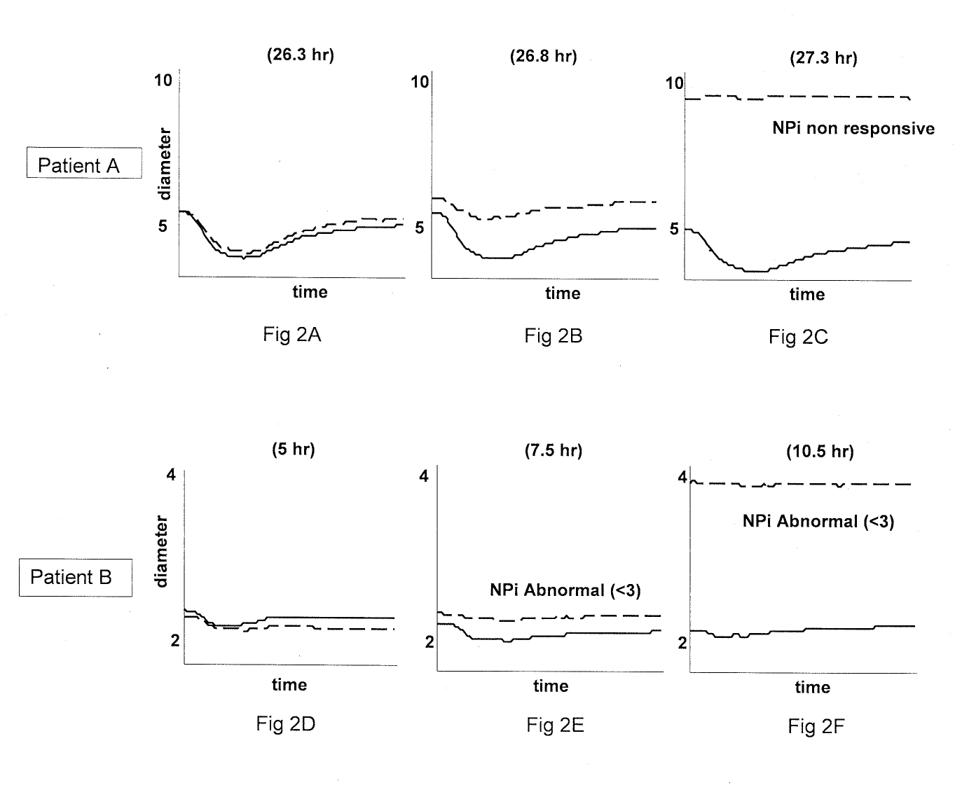

FIGS. 2A-2F show the temporal progression of the Neurological Pupil, Scalar value, index in two patients, Patient A and Patient B, illustrating how a scalar value for a patient can change over time. The left pupil is shown by the solid line and the right pupil is shown by the broken line. Each panel displays a pair of measurements for both pupils taken a few seconds apart. The time of acquisition is reported at the top of each panel and is expressed in hours relative to the time of patient enrollment (e.g., 26.3 hours from the beginning of the enrollment for patient A and 5 hours from the beginning of enrollment for patient B, FIGS. 2A and 2D). Initially both Patient A and Patient B had normally reactive pupils although both patients had a right pupil scalar value abnormality (FIGS. 2B and 2E). Over time, the right pupils in both patients not only remained abnormal (and patient A's pupil became actually non-responsive) but they also degenerated in a strong asymmetry (FIGS. 2C and 2F).

Patient A had larger pupils than Patient B and developed a Scalar value abnormality in the right pupil after half an hour (FIG. 2B). This abnormality was even more evident in the next half hour when the right pupil became non-reactive (FIG. 2C). Patient A had a right frontal aneurysmal subarachnoid hemorrhage. The CT scan performed at about the same time of the Scalar value deterioration revealed absent cisterns, a right to left midline shift of 8 mm and a mass size of 4 cc. ICP increased to over 50 mmHg. Cerebral ischemia was diagnosed ten hours later.

Patient B followed the same, although slower, course of deterioration for the right pupil (FIGS. 2E and 2F). Note how the abnormal pupils, in the middle, were still responsive for both patients and there was an insignificant difference in size between the left and right pupil. It is in the following acquisition that, the right pupil remained abnormal, and also developed a strong asymmetry with the left pupil. Patient B had TBI with an occipital parenchymal hemorrhage and a right lateral intraventricular hemorrhage. The CT scan revealed absent cisterns, no midline shift and a mass size of 4 cc (right sided). An increase of the intra-cranial volume accompanied by a raise of ICP above 50 mmHg was reported several hours later.

Patients were monitored continuously for 72 consecutive hours. Pupil measurements with the pupillometer were performed by the clinical research coordinator or the bedside nurse in groups of four consecutive measurements (right pupil—left pupil—right pupil—left pupil) every 30 minutes and ICP measurements were taken at the same time interval. Manual pupil measurements were also performed every hour using the pupil gauge for size and the pen flashlight for reactivity. Data from the pupillometer were not used in the treatment of the patients enrolled in the study. Manual pupil measurements were also performed every hour using the pupil gauge for size and a pen flashlight for reactivity. Manual and pupillometer results were compared only if they were taken within a few seconds of each other. Measurements were interrupted at any time a patient required medical or surgical intervention and thus some patients in the study underwent periods where no pupil information was available. All data from the pupillometer were downloaded to a computer and a scalar value algorithm was applied to the downloaded pupillary data. Pupillometer data and ICP data were recorded every half hour and collected in the same record along with any manual pupil measurements and any CT scan records performed at the same time.

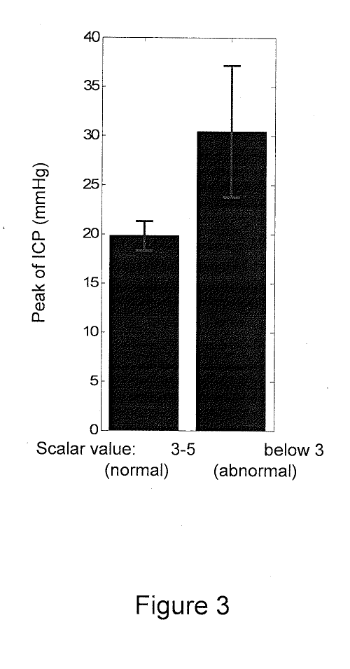

Each ICP measurement was averaged with the surrounding measurements in order to filter out sudden and temporally circumscribed events of high ICP and, thus, comparisons were focused on those events that lasted more than one single evaluation (different numbers of the surrounding measurements were tested and found not to affect the results). This is referred to as “sustained ICP.” Each patient was thus represented by the peak of sustained ICP. Peak of sustained ICP was defined for each single patient as the maximum event of sustained ICP; the distribution of peak of ICP varied depending on the scalar value. As shown in FIG. 3, those patients with a normal scalar value (3-5) had the lowest ICP; those with one or more occurrence of abnormal scalar value (below 3) had a significantly larger distribution of sustained ICP (p=0.0016). Error bars indicate 95% confidence interval (“CI”). Patients were grouped based on whether they received an abnormal Scalar value score at any point during the 72 hrs. A one-tailed students t-test was performed to test for group differences. Agreement between pupillometer and manual evaluations was also examined. Firstly, manual measurements of pupil size to those made using the pupillometer were examined. The agreement of pupil reactivity evaluation between nurses and the Scalar value score via Cohen's Kappa index was then explored. Nurses evaluated patients as either “Brisk”, “Sluggish” or “Nonreactive”. This rating was subsequently compared to the Scalar value scores and the analysis was stratified on pupil size.

Subjects whose pupil measurements scored in the abnormal range on the Scalar value scale at least once during the 72-hour period of the study were characterized by higher peaks of ICP than subjects whose Scalar value was always normal. Group 1 included patients with Scalar value always in the normal range (between 3 and 5.) Group 2 included patients with one or more occurrences of abnormal Scalar value (below 3.) We found that patients in Group 1 (normal Scalar value, n=106) had lower peak ICP with a mean significantly smaller than the patients in Group 2 (abnormal Scalar value, n=32) (μ(Group 1)=19.9 mmHg vs. μ(Group 2)=30.5 mmHg, p=0.0016). If we restrict Group 2 to those patients that not only had abnormal Scalar values but who also developed a nonreactive pupil (n=16), then the corresponding distribution of ICP is even higher, (ICP (μ(Group 3)=35.7 mmHg, p=0.003). In those subjects with abnormal Scalar value pupils, we found that the first event of abnormality occurred, on average, 15.9 hours prior to the time of the maximum ICP (95% CI=−28.56, −3 hr.).

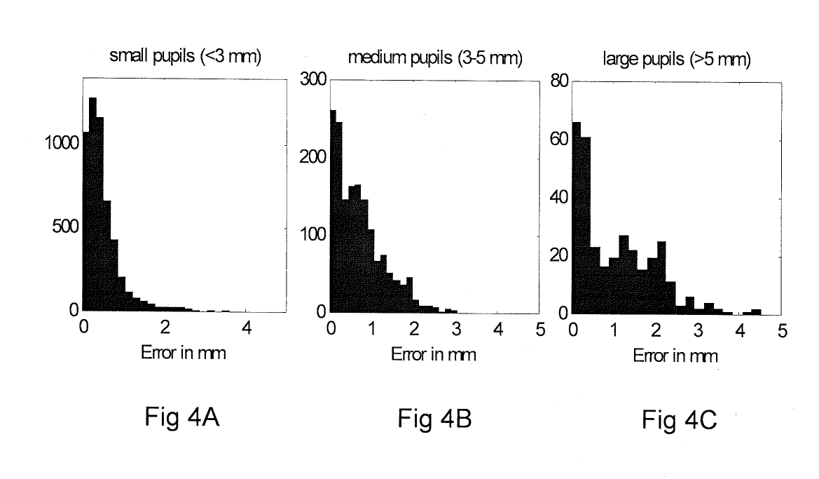

Pupillary data were divided into three major groups; small pupils (<3 mm), medium pupils (between 3 mm and 5 mm), and large pupils (>5 mm). In this study, 73.0% of all pupils were “small”, 22.4% of all pupils were “medium”, and only 4.6% were “large”. Manual examination was compared with pupillometer examination for each of these groups and resulted in different mean absolute errors; μ=0.49 mm (SD 0.42) for small pupils, μ=0.71 mm (SD 0.58) for medium pupils, μ=1.07 mm (SD 0.93) for large pupils. FIGS. 4A-4C show the distribution of human errors in evaluating pupil size for three different pupil size ranges. The mean measurement error was 0.5 mm (std 0.41) for small pupils; 0.7 mm (std 0.57) for medium pupils; 1.1 mm (std 0.58) for large pupils. Errors were evaluated by comparing measurements performed by the clinician with a pupil gauge and the Neuroptics pupillometer outcome for the same eye and patient. Pairs of measurements (human and pupillometer) were taken only a few seconds apart. The mean percentage of errors for small, medium, and large pupils were 21.4%, 18.7% and 19.0% respectively. This is similar to what was shown by Du et al. and Meeker et al.

Also important is the evaluation of the pupil light reflex. Manual examination was expressed using the customary classification of the pupil: “Brisk”, “Sluggish” and “Nonreactive” and compared with the Scalar value scale: “Normal” (scalar value between 3 and 5), “Abnormal” (Scalar value<3) and “Nonreactive.” Table 1 compares the clinicians' evaluations of pupillary reactivity to the scalar value classification of the patient and is broken down by small, medium, and large pupil size. This Table reports the number and percent or frequency of agreement between the clinician's rating of the pupils and the scalar value classification.

TABLE 1
Detection table for pupil reactivity; manual vs. the pupillometer Scalar

| Pupils smaller | | | kappa = 0.1 |
|---|---|---|---|
| than 3 mm | Normal | Abnormal | Non-Reactive |
| Nurse Brisk | 3,474 (71%) | 13 (39%) | 38 (15%) |
| Nurse Sluggish | 1,187 (24%) | 8 (24%) | 154 (61%) |
| Nurse Non-Reactive | 232 (5%) | 12 (36%) | 61 (24%) |
| Pupils within | | | kappa = 0.44 |
| 3 mm and 5 mm | Normal | Abnormal | Non-Reactive |
| Nurse Brisk | 1,232 (92%) | 55 (36%) | 6 (6%) |
| Nurse Sluggish | 103 (8%) | 61 (40%) | 62 (62%) |
| Nurse Non-Reactive | 1 (0%) | 37 (24%) | 32 (32%) |
| Pupils larger | | | kappa = 0.7 |
| than 5 mm | Normal | Abnormal | Non-Reactive |
| Nurse Brisk | 187 (99%) | 16 (27%) | 0 (0%) |
| Nurse Sluggish | 0 (0%) | 9 (15%) | 1 (1%) |
| Nurse Non-Reactive | 1 (1%) | 35 (58%) | 76 (99%) |

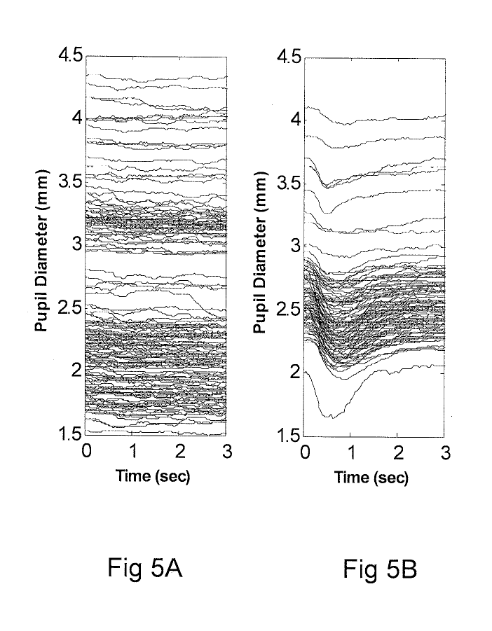

Manual examination of pupillary reactivity generally did not agree with the Scalar value rating. FIG. 5A shows examples of pupil profiles erroneously estimated and classified as reactive by clinicians. FIG. 5B shows examples of pupil profiles erroneously classified as non-reactive by the clinician. This is evidenced by the Cohen's kappa index which is a measure of inter-rater agreement for categorical data. A value of kappa greater than 0.75 indicates strong agreement beyond chance, values between 0.40 and 0.75 indicate fair to good agreement, and values below 0.40 indicate poor agreement. Kappa between the manual rating and the Scalar value rating was 0.1 for small pupils, 0.44 for medium pupils, and 0.7 for large pupils. The small pupil size range is where manual examination and Scalar value disagreed most. This is also the most commonly encountered clinical condition where analgesics and sedatives often induce a reduction of the pupil resting size (meiosis). In fact, 73% of our measurements and 100% of patients fell (entirely or temporarily) within this range during the study.

Pupil size and its light reflex mechanism is an integral part of the protocol for the treatment and management of severely head injured patients in intensive care units worldwide. The American Association of Neurological Surgeons and the Brain Trauma Foundation guidelines recommend that severe traumatic brain injury patients should be evaluated for asymmetry in pupil size or reactivity to light, as well as fixed and/or dilated pupils (3). The oculomotor nerve supplies efferent fibers to the extraocular muscles of the eye. Pupillomotor fibers travel along the dorsal periphery of the oculomotor nerve and are more sensitive to mass effect. The parasympathetic oculomotor nuclei in the midbrain are especially sensitive to brain stem compression and ischemia and thus, can indicate an expanding supra-tentorial mass lesion and onset of corresponding herniation. The TBI literature provides much evidence that alterations of the pupil light reflex, size of the pupil or anisocoria, are all closely correlated with the outcome of traumatic brain injury

When one considers the importance of pupil measurements, the fact that pupillary evaluation in the clinical setting is performed in a very subjective manner, with a pen flashlight for reactivity and a pupil gauge for pupil size, is paradoxical. These methods are affected by the examiner's subjectivity and by pronounced inter-examiner variability. The present study confirms that manual pupil examination by clinicians is often erroneous, especially when evaluating small pupils which are the most common size found in patients with severe TBI. The NeurOptics® device as used in the study is a hand held portable infrared pupillometer which allows a reliable and objective measurement of pupillary reflexes and pupil sizes. More importantly, the numeric scale of NPi™, a scalar value, allows a much more rigorous interpretation and classification of the pupil response. The notion of normal or abnormal pupil reflex is automatically derived by comparing the reflex against a scalar value which defines the behavior of the pupil mechanism. This removes any form of subjectivity from the measurement. The customary classification of the manual pupil examination, “brisk”, “sluggish” and “nonreactive” was compared against the scalar value for more than seven thousand measurements taken by the bedside nurses or site research coordinators and, as evidenced by Table 1, the manual evaluation was proved to be often incorrect.

In this study a group of patients who sustained severe intracranial injury were monitored for at least 72 hours in six different intensive care units and the results studied. ICP was continuously monitored. These patients were potentially severely compromised and admitted into the intensive care unit. It was found that patients who had one or more occurrences of abnormal pupil scalar value, were also characterized by peaks of sustained ICP significantly larger than those patients with normal pupillary activity. The scalar value was applied to patients with severe intracranial injuries and it was compared with manual pupil reactivity evaluations. Surprisingly, it was found that a scalar value was able to discriminate those patients with elevated peaks of ICP, i.e., patients with abnormal scalar value were characterized by peaks in ICP which were significantly higher than the peaks in ICP recorded in patients with a scalar value in the normal range. The results also show that despite the importance of examining the pupils of neurologically impaired patients, the measurement of pupil size and reactivity as reported during manual examination are often incorrect, especially for patients with small pupils which comprise most patients in this study. In view of the above, infrared pupillometry, which results in very accurate measurement of the pupil, and a scalar value which provides a more quantitative classification of the pupil light response, may be beneficial in the care and management of patients with severe intracranial insults.

While the invention is susceptible to various modifications and alternative forms, specific examples thereof have been shown by way of example in the drawings and are herein described in detail. It should be understood, however, that the invention is not to be limited to the particular forms or methods disclosed, but to the contrary, the invention is to cover all modifications, equivalents, and alternatives falling within the spirit and scope of the appended claims.

What is claimed is:

1. A pupillometer that is capable of receiving pupillary response data representative of one or more pupillary response characteristics of a patient, wherein the pupillometer comprises:
a microprocessor comprising an algorithm that converts the pupillary response data to a scalar value that indicates the patient's level of intracranial pressure.

2. The pupillometer of claim 1, further comprising a display for displaying the scalar value.

3. The pupillometer of claim 1, wherein the scalar value is represented by a numerical value, graphical depiction, color, sound, or other visual or audio means that indicates a value.

4. The pupillometer of claim 1, wherein the scalar value indicates that the patient's intracranial pressure is within a normal range, an abnormal range, or that the patient's pupil is non-responsive.

5. The pupillometer of claim 1, wherein the pupillary response data comprises pupillary latency.

6. The pupillometer of claim 1, wherein the pupillary response data comprises pupillary constriction velocity.

7. The pupillometer of claim 1, wherein the pupillary response data comprises pupillary first and second dilation velocity.

8. The pupillometer of claim 1, wherein the pupillary response data comprises pupillary amplitude.

9. The pupillometer of claim 1, wherein the pupillary response data comprises pupil diameter before stimulation and after recovery from stimulation.

10. The pupillometer of claim 1, wherein the pupillary response data comprises segmental dynamic analysis.

11. The pupillometer of claim 1, wherein the pupillary response data comprises segmental static analysis.

12. The pupillometer of claim 1, wherein the pupillary response data comprises pupillary recovery velocity.

13. The pupillometer of claim 1, wherein the pupillary response data comprises resting pupil diameter before stimulation.

14. The pupillometer of claim 1, wherein the pupillary response data comprises amount of constriction.

15. The pupillometer of claim 1, wherein the pupillary response data comprises pupillary latency, velocity of pupillary constriction, velocity of pupillary recovery, resting pupil size before constriction, and amount of constriction.

16. The pupillometer of claim 1, wherein the microprocessor is a stand-alone device separate from the pupillometer.

17. The pupillometer of claim 1, wherein the microprocessor is integrated in the pupillometer.

18. A diagnostic system for assessing the neurological status of a patient comprising:
a pupillometer that is capable of receiving pupillary response data representative of one or more pupillary response characteristics of the patient; and
a microprocessor in communication with the pupillometer, the microprocessor comprising an algorithm that converts the pupillary response data to a scalar value that indicates the patient's level of intracranial pressure.

19. The diagnostic system of claim 18, further comprising a display for displaying the scalar value.

20. The diagnostic system of claim 18, wherein the scalar value is represented by a numerical value, graphical depiction, color, sound, or other visual or audio means that indicates a value.

21. The diagnostic system of claim 18, wherein the scalar value indicates that the patient's intracranial pressure is within a normal range, an abnormal range, or that the patient's pupil is non-responsive.

22. The diagnostic system of claim 18, wherein the pupillary response data comprises pupillary latency.

23. The diagnostic system of claim 18, wherein the pupillary response data comprises pupillary constriction velocity.

24. The diagnostic system of claim 18, wherein the pupillary response data comprises pupillary first and second dilation velocity.

25. The diagnostic system of claim 18, wherein the pupillary response data comprises pupillary amplitude.

26. The diagnostic system of claim 18, wherein the pupillary response data comprises pupil diameter before stimulation and after recovery from stimulation.

27. The diagnostic system of claim 18, wherein the pupillary response data comprises segmental dynamic analysis.

28. The diagnostic system of claim 18, wherein the pupillary response data comprises segmental static analysis.

29. The diagnostic system of claim 18, wherein the pupillary response data comprises pupillary recovery velocity.

30. The diagnostic system of claim 18, wherein the pupillary response data comprises resting pupil diameter before stimulation.

31. The diagnostic system of claim 18, wherein the pupillary response data comprises amount of constriction.

32. The diagnostic system of claim 18, wherein the pupillary response data comprises pupillary latency, velocity of pupillary constriction, velocity of pupillary recovery, resting pupil size before constriction, and amount of constriction.

33. The diagnostic system of claim 18, wherein the microprocessor is a stand-alone device separate from the pupillometer.

34. The diagnostic system of claim 18, wherein the microprocessor is integrated in the pupillometer.

35. A method for assessing a patient's neurological status comprising:
using a pupillometer, obtaining pupillary response data from a patient, the pupillary response data being representative of one or more pupillary response characteristics of the patient;
providing a data analysis system comprising a microprocessor, the microprocessor in communication with the pupillometer, and the microprocessor comprising an algorithm that converts the pupillary response data to a scalar value;
using the data analysis system to derive a scalar value based on the pupillary response data from the patient, wherein the scalar value is indicative of the patient's level of intracranial pressure; and
assessing the neurological status of the patient based on the scalar value.

36. The method of claim 35, wherein the scalar value is represented by a numerical value, graphical depiction, color, sound, or other visual or audio means that indicates a value.

37. The method of claim 35, wherein the scalar value indicates that the patient's intracranial pressure is within a normal range, an abnormal range, or that the patient's pupil is non-responsive.

38. The method of claim 35, wherein the pupillary response data comprises pupillary latency.

39. The method of claim 35, wherein the pupillary response data comprises pupillary constriction velocity.

40. The method of claim 35, wherein the pupillary response data comprises pupillary first and second dilation velocity.

41. The method of claim 35, wherein the pupillary response data comprises pupillary amplitude.

42. The method of claim 35, wherein the pupillary response data comprises pupil diameter before stimulation and after recovery from stimulation.

43. The method of claim 35, wherein the pupillary response data comprises segmental dynamic analysis.

44. The method of claim 35, wherein the pupillary response data comprises segmental static analysis.

45. The method of claim 35, wherein the pupillary response data comprises pupillary recovery velocity.

46. The method of claim 35, wherein the pupillary response data comprises resting pupil diameter before stimulation.

47. The method of claim 35, wherein the pupillary response data comprises amount of constriction.

48. The method of claim 35, wherein the pupillary response data comprises pupillary latency, velocity of pupillary constriction, velocity of pupillary recovery, resting pupil size before constriction, and amount of constriction.

49. The method of claim 35, wherein the microprocessor is a stand-alone device separate from the pupillometer.

50. The method of claim 35, wherein the microprocessor is integrated in the pupillometer.
